SEQUENCE LISTING (4 sequences)

SEQ ID NO: 1 - DNA, 20 bp, Artificial Sequence
  Primer
ttccgggtcactgccatgga

SEQ ID NO: 2 - DNA, 22 bp, Artificial Sequence
  Primer
ccaggagagatgctgagggtgt

SEQ ID NO: 3 - DNA, 32 bp, Artificial Sequence
  Primer
gaagctcccagaatgccagaggctgctccccg

SEQ ID NO: 4 - DNA, 32 bp, Artificial Sequence
  Primer
gaagctcccagaatgccagaggctgctccccc

(19) United States
(12) Patent Application Publication
(10) Pub. No.: US 2005/0260607 A1
(43) Pub. Date: Nov. 24, 2005

(54) Single nucleotide polymorphism genotyping

(76) Inventors: Cheng Shiang Kao, Taipei County (TW); Shern Fwu Lee, Taipei City (TW); Yet Young Li, Changhua County (TW)

Correspondence Address: FISH & RICHARDSON PC, P.O. BOX 1022, MINNEAPOLIS, MN, 55440-1022, US

(21) Appl. No.: 10/851,408

(22) Filed: May 21, 2004

(51) Int. Cl. C12Q 1/68; C12P 19/34

(52) U.S. Cl. 435/6; 435/91.2

(57) ABSTRACT

A method of simultaneously identifying single nucleotide polymorphisms (SNPs) in a plurality of target regions.

### BACKGROUND

Single nucleotide polymorphisms (SNPs), a set of single nucleotide variants at genomic loci, are distributed throughout a genome.

An SNP can be “allelic.” More specifically, due to polymorphism, some members of a species have the unmutated sequence (i.e., the wild-type allele) and others have a mutated sequence (i.e., the mutant allele). In animals, a polymorphism may be associated with a genetic disorder. Examples of such a disorder include bovine leukocyte adhesion deficiency, citrullinemia, maple syrup urine disease, deficiency of uridine monophosphate synthase, a-mannosidosis, and generalized glycogenosis. In humans, an example of genetic disorders is cystic fibrosis, which affects about 0.05% of the entire Caucasian population. In addition, patients having different SNP genotypes respond to a treatment differently. Therefore, genome-wide SNP genotyping is expected to provide individualized guidance for preventing and treating these human disorders.

Current methods for identifying SNPs are tedious, slow, and costly, rendering genome-wise SNP genotyping unfeasible. Thus, there is a need for an efficient and inexpensive method.

### SUMMARY

This invention relates to an efficient and inexpensive SNP genotyping method, which allows one to simultaneously identify SNPs in a plurality of target regions of a subject. The method includes (i) obtaining nucleic acids from a subject, the nucleic acid including a plurality of target regions, each having a first nucleotide at an SNP site, a second nucleotide 3′ to the first nucleotide, and a first sequence 3′ to the second nucleotide; (ii) amplifying, by a polymerase chain reaction with amplification primers, the target regions to generate amplification products; (iii) annealing extension primers to the amplification products in a solution, each extension primer corresponds to each target region and has a first base that is located at the 3′ terminus of the extension primer and corresponds to the first nucleotide, a second base that corresponds to the second nucleotide, and a first segment that is complementary to the first sequence; (iv) incubating the solution to generate extension products, each extension product including a second segment that is complementary to a second sequence of the corresponding target region; (v) hybridizing the extension products to a nucleic acid array, each address of the array containing a capture probe that includes the second sequence of each target region; and (vi) monitoring a level of the hybridization of each address. If the hybridization level at an address is no less than a threshold level, the nucleotide at the SNP site in the target region corresponding to the address is determined to be complementary to the 3′ end nucleotide of the corresponding extension primer.

The just-described method can be used to identify SNPs in a plurality target regions, e.g., 1-100 or 30-50 regions. Preferably, the amplifying step is conducted under a low stringency condition. A “low stringency condition” refers to a PCR reaction condition that allows all primers to anneal to their respective target regions. It includes an annealing temperature that is low enough (e.g., 55 or 50° C.) or annealing temperatures that progressively decrease from a high temperature to a low temperature (e.g., decreasing by 1° C./cycle) as the PCR reaction proceeds. The resultant amplification products, which can be 50 to 3,000, e.g., 80-200, nucleotides in length, are then used in a primer extension reaction.

The just-mentioned extension reaction, which includes an annealing step and an incubating step, requires use of extension primers. These extension primers can have melting temperatures between 20 to 100° C. Their lengths can be between 10 and 50 nucleotides.

In the annealing step, the extension primers anneal to corresponding amplification products and serve as initiation points of primer extensions. In one embodiment, an extension primer contains one or more bases that mismatch the corresponding target region. For example, the second base in each extension primer is not complementary to the second nucleotide in the corresponding target region. This second based can be separated from the first base by 2-4 nucleotides.

In the incubating step, primer extensions are carried out. Preferably, these extensions are effected in a manner to generate extension products having a signal-producing label, and allowing one to identify and quantify the extension products. For example, it can be conducted in the presence of a nucleotide labeled with a fluorophore. In one embodiment, the extension primers contain (i) a first group of extension primers, the first base of each member being complementary to a wild type allele of the SNP site of each target region, and (ii) a second group of extension primers, the first base of each being complementary to a mutant allele of the SNP site of each target region. These two groups of extension primers are used in two independent incubating reactions, i.e., (i) a first reaction that contains the first group of extension primers and a nucleotide labeled with a first fluorophore to generate a first group of extension products, and (ii) a second reaction that contains the second group of extension primers and a nucleotide labeled with a second fluorophore to generate a second group of extension products. The first and second fluorophores (e.g., Cy3 and Cy5), upon excitation, emit lights of different wavelengths.

As the extension products have signal-producing labels, one can identify and quantify them on a nucleic acid array. Each address of the array has capture probes that are, preferably, 50 to 3,000 nucleotides in length and can hybridize to the extension products. For example, one can contact the nucleic acid array with the above-described first and second groups of extension products and determine the genotype of the subject based on, upon excitation of the first and second fluorophores, the intensities of the lights emitted by the two fluorophores at each address of the nucleic acid array.

The details of one or more embodiments of the invention are set forth in the accompanying description below. Other advantages, features, and objects of the invention will be apparent from the detailed description and the claims.

### DETAILED DESCRIPTION

This invention is based on an unexpected discovery that genomic DNA amplified by a multiplex PCR at low stringency can be used to simultaneously genotype SNPs of multiple regions. Accordingly, within the scope of this invention is an SNP genotyping method. This method is simple and rapid as it relies on high throughput multiplex PCR and microarray techniques. In addition, as a multiplex PCR can be conducted under a low stringency condition, the method does not require expensive polymerases.

To practice the method of this invention, one first obtains from a subject a test sample that contains genomic nucleic acids. Examples of the sample include tissues (e.g., hair or skin) or body fluids (e.g., blood or saliva). Then, a multiplexing PCR is conducted to partially amplify a plurality of DNA sequences from the samples under a low stringent condition (e.g., the annealing temperature being around 50-55° C. or progressively decreasing as the PCR reaction proceeds). The amplified sequences, which contain a plurality of target regions that include SNP sites to be typed, are then used in a primer extension reaction. It is known that, during a primer extension reaction, significant polymerization kinetic differences exist between matched and mismatched sites. When the 3′ terminal nucleotide of an extension primer matches a target nucleotide, the corresponding polymerization/primer extension speed is much faster than when it does not match. Thus, a matched extension primer allows for preferential (e.g., exclusive) production of a nucleic acid that contains a polymorphism. In other words, it can be used to preferentially producing one polymorphic allele (e.g., mutant allele) over the other (e.g., wild-type allele).

An extension primer corresponding to each target region can be designed based on the flanking sequence of a specific SNP site. For example, a target region has (i) a first nucleotide at an SNP site, (ii) a second nucleotide 3′ to the first nucleotide, and (iii) a first sequence 3′ to the second nucleotide. A corresponding extension primer can be designed to have (i) a first base that is located at its 3′ terminus and corresponds to the first nucleotide of the target region, (ii) a second base that corresponds to the second nucleotide of the target region, and (iii) a first segment that is 5′ to the second base and is complementary to the first sequence of the target region. After annealing to the target sequence, the primer serves as an initiation point for incorporating free nucleotides to synthesize a primer extension product. When the first nucleotide at an SNP site in a target nucleic acid is wild type, an extension primer containing a mutant base at its 3′ terminus has one mismatched base after annealing to the wild-type allele, as compared to annealing to a mutant allele. This extension primer is therefore not able to efficiently act as an initiation point for synthesis of a primer extension product. Conversely, the first nucleotide at an SNP site in a target nucleic acid is wild type, the extension primer perfectly matches the SNP site and acts as an efficient point of initiation. To facilitate such preferential production of a nucleic acid, one can artificially introduce more mismatches at positions −3 to −5 of the extension primer (i.e., the 3rd to the 5th nucleotides from the 3′ terminus) to achieve instability resulting from formation of a mis-matched primer-target region duplex.

A “primer,” either a PCR primer or an extension primer, mentioned above is an oligonucleotide capable of acting as a point of initiation of synthesis of a primer extension product that is complementary to a nucleic acid strand (template or target sequence), when placed under suitable conditions (e.g., salt concentration, temperature, and pH) in the presence of nucleotides and other reagents for nucleic acid polymerization (e.g., a DNA dependent polymerase). As known in the art, a primer must be of a sufficient length (e.g., at least 6 nucleotides) to prime the synthesis of extension products.

An extension primer can be optimized on a gene-by-gene basis to provide the greatest degree of discrimination between extension of the wild-type allele and the mutant allele. The optimization will of necessity include some empirical observations, but a number of basic principles can be applied to select a suitable starting point for optimization. An extension primer can be designed based on a known single nucleotide polymorphism in a gene, and also based on its properties, such as GC-content, annealing temperature, or internal pairing, all of which can be analyzed using software programs.

The above-described primer extension product contains a 3′ segment complementary to a sequence of the corresponding target region 5′ to the SNP site. This product allows one to hybridize the product to such a complementary target sequence immobilized on a solid substrate, e.g., a microarray, a for high throughput analysis. Therefore, the optimization of the extension primer may further take account of annealing or other properties of the two complementary sequences. To detect the amount of extension products, one can hybridize them to a nucleic acid array. Each address of this array contains a capture probe that includes the just-mentioned complementary target sequence. In one example, PCR products of each target region are spotted onto a specific address on a glass slide coated with poly-L-lysine. Various techniques and systems known in the art can be used to make the array.

Standard hybridization and monitoring techniques can be used to measure the hybridization level of each address. For example, the above-described primer extension can be conducted in the presence of nucleotides labeled with fluorophores or other detecting molecules. The resultant labeled products can be quantified by standard methods. One can also determine the product amount by measuring the conductivity of the nucleic acid molecules at each address according to the method described in Park S. J. et al., 2002, Science 295(5559):1503-6. Once nucleic acid duplexes formed at an address, the conductivity changes and can be quantified. If the hybridization level thus determined at an address is no less than a threshold level, the nucleotide at the SNP site in the target region corresponding to the address is determined to be complementary to the 3′ end nucleotide of the corresponding extension primer. A threshold level refers to a heuristically determined signal-to-noise ratio. As described in the example below, the threshold level can be at least 3 (e.g., 5 or 7) when the foreground and background fluorophore intensities of each address represent the signal and the noise, respectively. Such a threshold allows one to filter out unreliable signals. Alternatively, a threshold level can be determined based on other methods known in the art. See, e.g., that described in Chen et al., 2002, Bioinformatics. 18(9):1207-15.

As the identity of a capture probe spotted at each address is known, the array allows one to detect the amount of a corresponding extension product and identify the product without separating it from other products prior to the hybridization step. The hybridization step itself separates all extension products.

The above-described method uses high throughput multiplex PCR and microarray techniques. To increase specificity, it resorts to four layers of selection processes, i.e., PCR amplification, hybridization by extension primer, primer extension, and array hybridization. As the amplification step reduces the complexity of the samples, experimental artifacts due to cross-hybridization are minimized. To enhance signal/efficiency, the method of this invention relies on locus specific PCR, instead of whole genome amplification, to increase specificity. As a result, the method of this invention is more efficiently and cost-effectively than conventional methods.

The specific example below is to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLE

Genomic DNA was extracted from saliva collected from a subject. Fifty pairs of primers specific for 50 genes were designed and synthesized using standard techniques. The primer pairs were pooled together and used to amplify the corresponding alleles from 100 ng genomic DNA by multiplex PCR amplification In one case, a one-step multiplex PCR was carried out in a reaction buffer (containing 0.2 [M of each allele-specific primers, 0.5 μM dNTPs, 450 nM magnesium, 0.25 U of Taq DNA polymerase, and 1× Taq buffer). The PCR reaction was performed under a low stringent condition (40 cycles of: denaturing at 94° C. for 30 seconds, annealing at 50° C. for 30 seconds, and elongating at 72° C. for 1 minute).

In another case, a two-step multiplex PCR was conducted. First, in a primary multiplex PCR, more than 10 ng Genomic DNA was amplified with primary PCR primer pairs (200 nM each) in a tube containing 1.6×PCR buffer, 1 mM dNTPs, 3 mM MgCl2, 10 U Taq DNA ploymerase, and 10% DMSO. The PCR was performed under the following condition: (1) 94° C. for 5 minutes for denaturing; (2) 10 cycles of: 94° C. for 40 seconds, 65° C. for 30 seconds (decrease 1° C. for each cycle), and 72° C. for 1 minute 30 seconds; and (3) 30 cycles of: 94° C. for 40 seconds, 55° C. for 30 seconds, and 72° C. for 1 minute 30 seconds. Second, in a secondary PCR reaction, the primary PCR products thus obtained were diluted by 20 times in distilled water. At least 10 ng DNA was used as DNA templates. In this secondary multiplex PCR, the amplifications were performed using nested primer pairs, which were located within the primary PCR products to generate 50-3 kb PCR products (preferably, 80-200 bp) under the same conditions described in the primary multiplex PCR.

The products generated by the above-described one-step or two-step multiplex PCR were collected, cleansed up by ethanol precipitation, and then used as templates for primer extensions. In general, for each reaction, 5 ug of PCR products was mixed with 1×PCR buffer, 240 mM dATP, 240 mM dTTP, 240 mM dGTP, 120 mM dCTP, 120 mM Cy3/Cy5 labeled dCTP, 1.5 mM MgCl2, 2.5 U Taq DNA ploymerase, and 1-100 sets of 400 nM extension primers. Linear amplification was performed under following conditions: (1) 94° C. for 5 minutes for denaturing; (2) 30 cycles of: 94° C. for 30 seconds, 72° C. for 30 seconds (decrease 0.3° C. for each cycle), and 72° C. for 30 seconds; and (3) 72° C. for 7 minutes. Two independent multiplex extension reactions were carried out in two tubes. One contained mutant extension primers and Cy5-labeled dCTPs. The other contained wild type extension primers and Cy3-labeled dCTPs. Cy-5 and Cy3 are two dyes that, upon being excited by lights of 635 nm and 532 nm, emit lights of 670 nm and 570 nm, respectively. After the reactions, free dyes were removed from the extension products by QuiaQucik PCR clean-up kit (Quiagene).

In the above-described multiplex PCR amplification and primer extension, Taq DNA polymerase, instead of Klenow or Phi-29 DNA polymerase, was used. Further, use of dye-tagged dNTP, instead of dye-tagged ddNTP, resulted in much stronger signals due to the much higher incorporation rates of dye-tagged dNTPs into the extension products.

The purified extension products mentioned above were dried in a spin vacuum, re-hydrided by mixing 25 μl hybridization buffer, which contained 3×SSC and 0.2% SDS, and denatured at 100° C. for 2 minutes. Microarrays were made on poly-L-lysine coated glass slides by standard techniques. Hybridization was carried out at 63° C. for 16 hours in a humidified chamber dispensed with 3×SSC. The array slides were washed sequentially with 1×SSC and 0.03% SDS for 10 minutes, 0.2×SSC for 20 minutes, 0.05×SSC for 10 minutes, and finally rinsed with ddH₂O. They were dried by spinning off the water at 1000 rpm for 5 minutes in a Centrifuge Sorvall RT7.

Hybridization signals for at each address were determined by an Axon florescence scanner (Axon Instruments, Inc., Union City, Calif.). More specifically, the signals resulted from the hybridization between mutant extension products and the capture probes, and between wild type extension products and the capture probes were measured via the 635 nm and 532 nm channels, respectively.

Four different plant genes were used as spiked-in controls (C₁]-14) for heterozygous genotypes. More specifically, 150 ng, 300 ng, 600 ng and 1 mg of PCR products (50-3,000 bps) from these plant genes were used to carry out primer extension in the presence of both types of the above-mentioned dyes, respectively. 500 nM of corresponding extension primers (10-50 mer) was added to the extension reactions. Each primer perfectly matched the corresponding template and was designed to locate in the middle region of each PCR product. The resultant extension products had both dyes in equal amounts and were detectable via both the 635 nm and 532 mm channels. Thus, they were used to represent extension products from heterozygous subjects. Briefly, the signals thus obtained (i.e., F635, F532, B635, and B532; F for foreground and B for background), were used to determine the genotype based on the rationale mentioned in Ahmadian et al., 2001, Nucleic Acid Research, 29, e121. An allelic fraction was computed as the relative proportion of the normalized intensities of each SNP detection primer using the following formula:
AF(Allelic fraction)=X/(X+Y),
X=Absolute intensities of Wild type channel,
Y=Absolute intensities of Mutant channel.

As the spiked-in controls had equal amounts of two different products, their “allelic fraction” should be 0.5, i.e., heterozygous genotype. The result indicated that they had an average allelic fraction (AF) of 0.74. The standard deviation of each spiked-in “heterozygote” was computed out as 0.1 and used to set up the upper and lower boundaries for heterozygous clusters. More specifically, the boundaries for heterozygous clusters ranged from plus 3-fold to minus 3-fold of the standard deviation from the expected allelic fraction of 0.5 (i.e., 0.5±3×0.1), or between 0.2 and 0.8. Based on these boundaries, 11 of the 50 genes were typed. The results were listed in the table below.

| | Average Of | Average Of | | | Allelic | | Genotype |
|---|---|---|---|---|---|---|---|
| Name | F635-B635 | F532-B532 | SD of 635 | SD of 532 | Fraction | Note | Defined |
| A11 | 62.5 | 796 | 6.244998 | 43.703547 | 0.0728014 | | Wild type |
| A12 | 1.5 | 34.25 | 1.2909944 | 2.6299556 | 0.0419580 | signal too weak | NA |
| A13 | 46.75 | 113.5 | 7.9320027 | 16.217275 | 0.2917317 | signal too weak | NA |
| A14 | 30.5 | 352.75 | 4.7958315 | 34.893887 | 0.0795825 | | Wild type |
| A15 | 452.5 | 326 | 15.154757 | 8.7559504 | 0.5812460 | | Heterozygote |
| A16 | 572 | 302.75 | 26.267851 | 16.640813 | 0.6539011 | | Heterozygote |
| A17 | 283 | 40.25 | 24.124676 | 2.6299556 | 0.8754834 | | Mutant |
| A18 | 235.5 | 58 | 24.610296 | 5.5976185 | 0.8023850 | | Mutant |
| A21 | 6.25 | 400.75 | 0.5 | 16.760569 | 0.0153563 | | Wild type |
| A22 | 126.5 | 116.5 | 44.873897 | 27.16002 | 0.5205761 | | Heterozygote |
| A23 | 165.75 | 351 | 11.58663 | 29.086079 | 0.3207547 | | Heterozygote |
| C11 | 264.25 | 97.25 | 33.290389 | 7.410578 | 0.7309820 | | |
| C12 | 22.5 | 17.25 | 2.3804761 | 3.3040379 | 0.5660377 | | |
| C13 | 324 | 98.5 | 58.793424 | 15.84298 | 0.7668639 | | |
| C14 | 376.75 | 145.75 | 55.811438 | 33.430276 | 0.7210526 | | |

One of the above-listed genes, A21, was the tumor repressor p53 gene. The polymorphism of PRO72ARG transition was studied. The primers were A21PCR-F: TTCCGGGTCACTGCCATGGA (Tm=67° C.) and A21 PCR-R: CCAGGAGAGATGCTGAGGGTGT (Tm=67° C.). The wild type and mutant extension primers were A21SNP-WT: gaagctcccagaatgccagaggctgctccccG (Tm=67° C.) and A21 SNP-M (gaagctcccagaatgccagaggctgctcccC Tm=67° C.). In this case, the Arg/Arg, Pro/Pro, and Pro/Arg genotypes were defined as wild type, mutant genotype, and heterozygous type, respectively.

The above results were confirmed by direct DNA sequencing, proving that the method described in this example can be used in SNP typing multiple loci simultaneously.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1. A method of simultaneously identifying single nucleotide polymorphisms in a plurality of target regions, the method comprising:
obtaining nucleic acids from a subject, the nucleic acid including a plurality of target regions, each having a first nucleotide that is at an SNP site, a second nucleotide 3′ to the first nucleotide, and a first sequence that is 3′ to the second nucleotide;
amplifying, by a polymerase chain reaction with amplification primers, the target regions to generate amplification products;
annealing extension primers to the amplification products in a solution, wherein each extension primer corresponds to each target region and has a first base that is located at the 3′ terminus of the extension primer and corresponds to the first nucleotide, a second base that corresponds to the second nucleotide, and a first segment that is complementary to the first sequence;
incubating the solution to generate extension products, each extension product including a second segment that is complementary to a second sequence of the corresponding target region;
hybridizing the extension products to a nucleic acid array, each address of the array containing a capture probe that includes the second sequence of each target region; and
monitoring a level of the hybridization of each address, whereby, if the hybridization level at an address is no less than a threshold level, the nucleotide at the SNP site in the target region corresponding to the address is determined to be complementary to the 3′ end nucleotide of the corresponding extension primer.

2. The method of claim 1, wherein the nucleic acids include 1-100 target regions.

3. The method of claim 2, wherein the nucleic acids include 30-50 target regions.

4. The method of claim 3, wherein the extension primers have melting temperatures between 20 to 100° C.

5. The method of claim 4, wherein the amplifying step is effected under a low stringency condition.

6. The method of claim 5, wherein the extension primers are 10-50 nucleotides in length.

7. The method of claim 6, wherein the amplification products are 50-3,000 nucleotides in length.

8. The method of claim 7, wherein the capture probes are 50-3,000 nucleotides in length.

9. The method of claim 8, wherein the incubating step is effected in a manner to generate extension products having labels and the monitoring step is effected by detecting a signal based on the labels.

10. The method of claim 9, wherein the incubating step is effected in presence of a nucleotide labeled with a fluorophore.

11. The method of claim 4, wherein the extension primers are 10-50 nucleotides in length.

12. The method of claim 4, wherein the amplification products are 50-3,000 nucleotides in length.

13. The method of claim 4, wherein the capture probes are 50-3,000 nucleotides in length.

14. The method of claim 4, wherein the incubating step is effected in a manner to generate extension products having labels and the monitoring step is effected by detecting a signal based on the labels.

15. The method of claim 14, wherein the incubating step is effected in presence of a nucleotide labeled with a fluorophore.

16. The method of claim 3, wherein the amplifying step is effected under a low stringency condition.

17. The method of claim 3, wherein the extension primers are 10-50 nucleotides in length.

18. The method of claim 3, wherein the amplification products are 50-3,000 nucleotides in length.

19. The method of claim 3, wherein the capture probes are 50-3,000 nucleotides in length.

20. The method of claim 3, wherein the incubating step is effected in a manner to generate extension products having labels and the monitoring step is effected by detecting a signal based on the labels.

21. The method of claim 20, wherein the incubating step is effected in presence of a nucleotide labeled with a fluorophore.

22. The method of claim 1, wherein the extension primers have melting temperatures between 20 to 100° C.

23. The method of claim 22 wherein the amplifying step is effected under a low stringency condition.

24. The method of claim 22, wherein the extension primers are 10-50 nucleotides in length.

25. The method of claim 22, wherein the amplification products are 50-3,000 nucleotides in length.

26. The method of claim 22, wherein the capture probes are 50-3,000 nucleotides in length.

27. The method of claim 22, wherein the incubating step is effected in a manner to generate extension products having labels and the monitoring step is effected by detecting a signal based on the labels.

28. The method of claim 27, wherein the incubating step is effected in presence of a nucleotide labeled with a fluorophore.

29. The method of claim 1, wherein the amplifying step is effected under a low stringency condition.

30. The method of claim 29, wherein the extension primers are 10-50 nucleotides in length.

31. The method of claim 29, wherein the amplification products are 50-3,000 nucleotides in length.

32. The method of claim 29, wherein the capture probes are 50-3,000 nucleotides in length.

33. The method of claim 29, wherein the incubating step is effected in a manner to generate extension products having labels and the monitoring step is effected by detecting a signal based on the labels.

34. The method of claim 34, wherein the incubating step is effected in presence of a nucleotide labeled with a fluorophore.

35. The method of claim 1, wherein the extension primers are 10-50 nucleotides in length.

36. The method of claim 1, wherein the amplification products are 50-3,000 nucleotides in length.

37. The method of claim 36, wherein the amplification products are 80-200 nucleotides in length.

38. The method of claim 1, wherein the capture probes are 50-3,000 nucleotides in length.

39. The method of claim 1, wherein the incubating step is effected in a manner to generate extension products having labels and the monitoring step is effected by detecting a signal based on the labels.

40. The method of claim 39, wherein the incubating step is effected in presence of a nucleotide labeled with a fluorophore.

41. The method of claim 1, wherein the second base is not complementary to the second nucleotide.

42. The method of claim 41, wherein the second base is separated from the first base by 2-4 nucleotides.

43. The method of claim 1, wherein the extension primers contain a first group of extension primers, the first base of each member being complementary to a wild type allele of the SNP site of each target region, and a second group of extension primers, the first base of each being complementary to a mutant allele of the SNP site of each target region.

44. The method of claim 43, wherein the incubating step is effected by conducting a first reaction that contains the first group of extension primers and a nucleotide labeled with a first fluorophore to generate a first group of extension products, and a second reaction that contains the second group of extension primers and a nucleotide labeled with a second fluorophore to generate a second group of extension products, the first and second reactions being conducted independently.

45. The method of claim 44, wherein the hybridizing step is effected by contacting the nucleic acid array with the first group of extension products and the second group of extension products, whereby the genotype of the subject is determined based on, upon excitation of the first and second fluorophores, the intensities of the lights emitted by the two fluorophores at each address of the nucleic acid array.
